(19) United States
(12) Patent Application Publication
(10) Pub. No.: US 2003/0004148 A1
(43) Pub. Date: Jan. 2, 2003

(54) Regulation of meiosis

(76) Inventors: Anne G. Byskov, Gentofte (DK); Claus Y. Andersen, Copenhagen (DK); Lars Nordholm, Herlev (DK); Henning Thogersen, Farum (DK); Ole Wassman, Vallensbak (DK); Ivan Verner Diers, Varlose (DK); Erling Guddahl, Brondby (DK)

Correspondence Address: Reza Green, Esq., Novo Nordisk of North America, Inc., Suite 6400, 405 Lexington Avenue, New York, NY, 10174-6401, US

(21) Appl. No.: 10/058,436

(22) Filed: Jan. 23, 2002

Related U.S. Application Data
(63) Continuation-in-part of application No. 09/440,590, filed on Nov. 15, 1999, now abandoned.
Application No. 09/440,590 is a continuation-in-part of application No. 09/017,087, filed on Feb. 2, 1998, now abandoned.
Application No. 09/017,087 is a continuation of application No. 08/448,217, filed on May 23, 1995, now Pat. No. 5,716,777.

(30) Foreign Application Priority Data
Jun. 23, 1994 (DK) 0753/94
Mar. 9, 1995 (DK) 0241/95

(51) Int. Cl. A61K 31/56

(52) U.S. Cl. 514/182

(57) ABSTRACT

Compounds of Formula I and methods of regulating the meiosis in a mammalian germ cell which method comprises administering an effective amount of the compound of Formula I to a germ cell in need of such a treatment.

### RELATED APPLICATIONS

This application is a continuation-in-part of application Ser. No. 09/440,590, filed Nov. 16, 1999, which is a continuation-in-part application to U.S. Ser. No. 09/017,087 filed Feb. 2, 1998, which is a continuation of U.S. Ser. No. 08/448,217 fled May 23, 1995, the contents of which are herein incorporated by reference. This application also claims priority under 35 USC 119 to Danish patent applications 0753/94 filed Jun. 23, 1994 and 0241/95 filed Mar. 9, 1995, the contents of which are also herein incorporated by reference.

### FIELD OF THE INVENTION

The present invention relates to certain sterol derivatives and to their use as medicaments. More particularly it has been found that certain sterol derivatives can be used for regulating the meiosis.

### BACKGROUND OF THE INVENTION

Meiosis is the unique and ultimate event of germ cells on which sexual reproduction is based. Meiosis comprises two meiotic divisions. During the first division, exchange between maternal and paternal genes take place before the pairs of chromosomes are separated into the two daughter cells. These contain only half the number (1n) of chromosomes and 2c DNA. The second meiotic division proceeds without a DNA synthesis. This division therefore results in the formation of the haploid germ cells with only 1c DNA.

The meiotic events are similar in the male and female germ cells, but the time schedule and the differentiation processes which lead to ova and to spermatozoa differ profoundly. All female germ cells enter the prophase of the first meiotic division early in life, often before birth, but all are arrested as oocytes later in the prophase (dictyatestate) until ovulation after puberty. Thus, from early life the female has a stock of oocytes which is drawn upon until the stock is exhausted. Meiosis in females is not completed until after fertilization, and results in only one ovum and two abortive polar bodies per germ cell. In contrast, only some of the male germ cells enter meiosis from puberty and leave a stem population of germ cells throughout life. Once initiated, meiosis in the male cell proceeds without significant delay and produces 4 spermatozoa.

Only little is known about the mechanisms which control the initiation of meiosis in the male and in the female. In the oocyte, new studies indicate that follicular purines, hypoxanthine or adenosine, could be responsible for meiotic arrest (Downs, S. M. et al. Dev. Biol. 82 (1985) 454-458; Eppig, J. J. et al. Dev. Biol. 119 (1986) 313-321; and Downs, S. M. Mol. Reprod. Dev. 35 (1993) 82-94). The presence of a diffusible meiosis regulating substance was first described by Byskov et al. in a culture system of fetal mouse gonads (Byskov, A. G. et al. Dev. Biol. 52 (1976) 193-200). A meiosis inducing substance (MIS) was secreted by the fetal mouse ovary in which meiosis was ongoing, and a meiosis preventing substance (MPS) was released from the morphologically differentiated testis with resting, non-meiotic germ cells. It was suggested that the relative concentrations of MIS and MPS regulated the beginning, arrest and resumption of meiosis in the male and in the female germ cells (Byskov, A. G. et al. in The Physiology of Reproduction (eds. Knobil, E. and Neill, J. D., Raven Press, New York (1994)). Clearly, if meiosis can be regulated, reproduction can be controlled. Unfortunately, up till now it has not been possible to identify a meiosis inducing substance.

### SUMMARY OF THE INVENTION

It has surprisingly been found that certain sterols known as intermediates in the biosynthesis of cholesterol and some novel, structurally related synthetic sterols can be used for regulating the meiosis.

Accordingly, the present invention relates to a compound of the general formula (I)

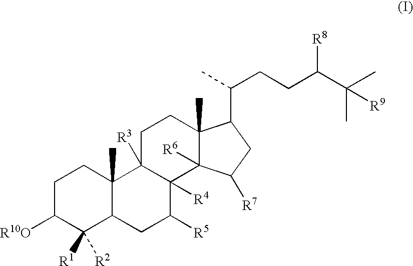

wherein R¹ and R², independently, are selected from the group comprising hydrogen, unbranched or branched C₁-C₆ alkyl which may be substituted by halogen or hydroxy or wherein R¹ and R² together with the carbon atom to which they are bound form a cyclopentane ring or a cyclohexane ring;

R³ and R⁴ together designate an additional bond between the carbon atoms to which they are bound in which case R⁵ is hydrogen and R⁶ and R⁷ are either hydrogen or together they designate an additional bond between the carbon atoms to which they are bound; or

R⁵ and R⁴ together designate an additional bond between the carbon atoms to which they are bound in which case R³ is hydrogen and R⁶ and R⁷ are either hydrogen or together they designate an additional bond between the carbon atoms to which they are bound; or

R⁶ and R⁴ together designate an additional bond between the carbon atoms to which they are bound in which case R³, R⁵ and R⁷ are all hydrogen;

R⁸ and R⁹ are hydrogen or together they designate an additional bond between the carbon atoms to which they are bound; and

R¹⁰ is either hydrogen, an acyl group, a phosphono group or a sulfo group or R¹⁰ is a group which together with the remaining part of the molecule forms an ether, for use as a medicament.

In a further aspect, the present invention relates to novel compounds of the general formula (I).

In the present context, the expression “regulating the meiosis” is understood to indicate that the compounds can be used for stimulating the meiosis in vitro, in vivo, and ex vivo.

Accordingly, in a more specific aspect, the present invention relates to the use of a compound of general formula (I) above in the regulation of the meiosis.

In a still further aspect, the present invention relates to a method of regulating the meiosis in a mammalian germ cell which method comprises administering an effective amount of a compound of the general formula (I) above to a germ cell in need of such a treatment.

### BRIEF DESCRIPTION OF THE DRAWING

FIG. 1 shows the effect of LH, FF-MAS and cholesterol on GVB in isolated perfused ovaries from immature rats. Asteriks indicate significant differences (p<0.05) compared to control.

### DETAILED DESCRIPTION OF THE INVENTION

It has been found, that the meiosis inducing substances extracted from bull testes and from human follicular fluid are both able to induce resumption of meiosis in cultured mouse oocytes (the oocyte test) and also to stimulate the meiosis in male germ cells of cultured fetal mouse testes (the gonad test). A meiosis inducing substance is produced by adult testes of various mammals, including man, and is also found in mature ovarian follicles of several mammalian species, including women. As it appears from the Examples 1 and 2, the meiosis inducing substance found in but testes is 4,4-dimethylzymosterol while the meiosis inducing substance found in human follicular fluid is 4,4-dimethyl-5α-cholesta-8,14,24-triene-3β-ol.

The existence of a meiosis inducing substances has been known for some time. However, until now the identity of the meiosis inducing substance or substances has been unknown. To the best of the knowledge of the present inventors, no practical use of the compounds of the general formula (I) has so far been made in medicine. In particular, no compounds of the general formula (I) have so far been used as medicaments for regulating the meiosis.

The prospects of being able to influence the meiosis are several. In a preferred embodiment of the present invention, the compounds of the general formula (I) are used to stimulate the meiosis. In another preferred embodiment of the present invention, the compounds of the general formula (I) are used to stimulate the meiosis in humans. Thus, the compounds of formula (I) are promising as new fertility regulating agents without the usual side effect on the somatic cells which are known from the hitherto used hormonal contraceptives which are based on estrogens and/or gestagens. For use as a contraceptive agent in females, a meiosis inducing substance can be administered so as to prematurely induce resumption of meiosis in oocytes while they are still in the growing follicle, before the ovulatory peak of gonadotropins occurs. In women, the resumption of the meiosis can, for example, be induced a week after the preceding menstruation has ceased. When ovulated, the resulting overmature oocytes are most likely not to be fertilized. The normal menstrual cycle is not likely to be affected. In this connection it is important to notice, that the biosynthesis of progesterone in cultured human granulosa cells (somatic cells of the follicle) is not affected by the presence of a meiosis inducing substance whereas the estrogens and gestagens used in the hitherto used hormonal contraceptives do have an adverse effect on the biosynthesis of progesterone.

In another aspect of this invention, a meiosis inducing substance of the general formula (I) can be used in the treatment of certain cases of infertility in females, including women, by administration thereof to females who, due to an insufficient own production of MIS, are unable to produce mature oocytes. Also, when in vitro fertilization is performed, better results are achieved, when a meiosis inducing substance of the general formula (I) is added to the medium in which the oocytes are kept.

Also, when infertility in males, including men, is caused by an insufficient own production of the meiosis inducing substance administration of a meiosis inducing substance of the general formula (I) may relieve the problem.

The route of administration of the compositions containing a compound of formula (I) may be any route which effectively transports the active compound to its site of action.

Thus, when the compounds of this invention are to be administered to a mammal, they are conveniently provided in the form of a pharmaceutical composition which comprises at least one compound of formula (I) in connection with a pharmaceutically acceptable carrier. In a specific embodiment, a dosage of about 1 to about 10 g per day is administered to the mammal. For oral use, such compositions are preferably in the form of capsules or tablets.

From the above it will be understood that administrative regimen called for will depend on the condition to be treated. Thus, when used in the treatment of infertility the administration may be once only, or for a limited period, e.g. until pregnancy is achieved. When used as a contraceptive, the meiosis inducing substance of the general formula (I) will either have to be taken continuously or cyclically. When used as a contraceptive by women and not taken continuously, the timing relative to the menstrual cycle will be important.

The pharmaceutical compositions may comprise carriers, diluents, absorption enhancers, preservatives, buffers, agents for adjusting the osmotic pressure, tablet disintegrating agents and other ingredients which are conventionally used in the art. Examples of solid carriers are magnesium carbonate, magnesium stearate, dextrin, lactose, sugar, talc, gelatin, pectin, tragacanth, methyl cellulose, sodium carboxymethyl cellulose, low melting waxes and cocoa butter.

Liquid compositions include sterile solutions, suspensions and emulsions. Such liquid compositions may be suitable for injection or for use in connection with ex vivo and in vitro fertilization. The liquid compositions may contain other ingredients which are conventionally used in the art, some of which are mentioned in the list above.

Further, a composition for transdermal administration of a compound of this invention may be provided in the form of a patch and a composition for nasal administration may be provided in the form of a nasal spray in liquid or powder form.

The dose of a compound of the invention to be used will be determined by a physician and will depend, inter alia, on the particular compound employed, on the route of administration and on the purpose of the use.

Preferred compounds of formula (I) are the following:

Cholest-7-ene-^{3β}-ol;

4-Methylcholest-7-ene-^{3β}-ol;

4-Ethylcholest-7-ene-^{3β}-ol;

4,4-Dimethylcholest-7-ene-^{3β}-ol;

^{4α}-Methyl-4^{β}-ethylcholest-7-ene-^{3β}-ol;

^{4α}-Ethyl-4^{β}-methylcholest-7-ene-^{3β}-ol;

4,4-Diethylcholest-7-ene-^{3β}-ol;

4-Propylcholest-7-ene-^{3β}-ol;

4-Butylcholest-7-ene-^{3β}-ol;

4-Isobutylcholest-7-ene-^{3β}-ol;

4,4-Tetramethylenecholest-7-ene-^{3β}-ol;

4,4-Pentamethylenecholest-7-ene-^{3β}-ol;

Cholest-8-ene-^{3β}-ol;

4-Methylcholest-8-ene-^{3β}-ol;

4-Ethylcholest-8-ene-^{3β}-ol;

4,4-Dimethylcholest-8-ene-^{3β}-ol;

^{4α}-Methyl-4^{β}-ethylcholest-8-ene-^{3β}-ol;

^{4α}-Ethyl-4^{β}-methylcholest-8ene-^{3β}-ol;

4,4-Diethylcholest-8-ene-^{3β}-ol;

4-Propylcholest-8-ene-^{3β}-ol;

4-Butylcholest-8-ene-^{3β}-ol;

4-Isobutylcholest-8-ene-^{3β}-ol;

4,4-Tetramethylenecholest-8-ene-^{3β}-ol;

4,4-Pentamethylenecholest-8-ene-^{3β}-ol;

Cholest-8(14)-ene-^{3β}-ol;

4-Methylcholest-8(14)-ene-^{3β}-ol;

4-Ethylcholest-8(14)-ene-^{3β}-ol;

4,4-Dimethylcholest-8(14)-ene-3-ol;

^{4α}-Methyl-4^{β}-ethylcholest-8( 14)-ene-^{3β}-ol;

^{4α}-Ethyl-4^{β}-methylcholest-8(14)-ene-^{3β}-ol;

4,4-Diethylcholest-8(14)-ene-^{3β}-ol;

4-Propylcholest-8(14)-ene-^{3β}-ol;

4-Butylcholest-8(14)-ene-^{3β}-ol;

4-Isobutylcholest-8(14)-ene-^{3β}-ol;

4,4-Tetramethylenecholest-8(14)-ene-^{3β}-ol;

4,4-Pentamethylenecholest-8(14)-ene-^{3β}-ol;

Cholesta-8,14-diene-^{3β}-ol;

4-Methylcholesta-8,14-diene-^{3β}-ol;

4-Ethylcholesta-8,14-diene-^{3β}-ol;

4,4-Dimethylcholesta-8,14-diene-^{3β}-ol;

^{4α}-Methyl-4^{β}-ethylcholesta-8,14-diene-^{3β}-ol;

^{4α}-Ethyl-4^{β}-methylcholesta-8,14-diene-^{3β}-ol;

4,4-Diethylcholesta-8,14-diene-^{3β}-ol;

4-Propylcholesta-8,14-diene-^{3β}-ol;

4-Butylcholesta-8,14-diene-^{3β}-ol;

4-Isobutylcholesta-8,14-diene-^{3β}-ol;

4,4-Tetramethylenecholesta-8,14-diene-^{3β}-ol;

4,4-Pentamethylenecholesta-8,14-diene-^{3β}-ol;

Cholesta-8,24-diene-^{3β}-ol;

4-Methylcholesta-8,24-diene-^{3β}-ol;

4-Ethylcholesta-8,24-diene-^{3β}-ol;

4,4-Dimethylcholesta-8,24-diene-^{3β}-ol;

^{4α}-Methyl-4^{β}-ethylcholesta-8,24-diene-^{3β}-ol;

^{4α}-Ethyl-4^{β}-methylcholesta-8,24-diene-^{3β}-ol;

4,4-Diethylcholesta-8,24-diene-^{3β}-ol;

4-Propylcholesta-8,24-diene-^{3β}-ol;

4-Butylcholesta-8,24-diene-^{3β}-ol;

4-Isobutylcholesta-8,24-diene-^{3β}-ol;

4,4-Tetramethylenecholesta-8,24-diene-^{3β}-ol;

4,4-Pentamethylenecholesta-8,24-diene-^{3β}-ol;

Cholesta-8,14,24-triene-^{3β}-ol;

4-Methylcholesta-8,14,24-triene-^{3β}-ol;

4-Ethylcholesta-8,14,24-triene-^{3β}-ol;

4,4-Dimethylcholesta-8,14,24-triene-^{3β}-ol;

^{4α}-Methyl-4^{β}-ethylcholesta-8,14,24-triene-^{3β}-ol;

^{4α}-Ethyl-4^{β}-methylcholesta-8,14,24-triene-^{3β}-ol;

4,4-Diethylcholesta-8,14,24-triene-^{3β}-ol;

4-Propylcholesta-8,14,24-triene-^{3β}-ol;

4-Butylcholesta-8,14,24-triene-^{3β}-ol;

4-Isobutylcholesta-8,14,24-triene-^{3β}-ol;

4,4-Tetramethylenecholesta-8,14,24-triene-^{3β}-ol; and

4,4-Pentamethylenecholesta-8,14,24-triene-^{3β}-ol;

and esters and ethers thereof.

Preferred esters of formula (I) are those in which R¹⁰ is the acyl group of a carboxylic acid which may be branched or unbranched or cyclic and may comprise an optionally substituted amino group and/or 1 or 2 oxygen atoms further to the carbonyl oxygen of the ester group which links R¹⁰ to the sterol skeleton. When R¹⁰ designates an acyl group, it preferably comprises from 1 to 20 carbon atoms, more preferred from 1 to 12 carbon atoms, still more preferred from 1 to 10 carbon atoms, yet still more preferred from 1 to 7 carbon atoms. The acid from which R¹⁰ is derived may be a dicarboxylic acid. Examples of R¹⁰ are: acetyl, benzoyl, pivaloyl, butyryl, nicotinoyl, isonicotinoyl, hemi succinoyl, hemi glutaroyl, hemi maloyl, hemi phthaloyl, butylcarbamoyl, phenylcarbamoyl, butoxycarbonyl, tert-butoxycarbonyl, ethoxycarbonyl, 4-dimethylaminomethylbenzoyl, 4-diethylaminomethylbenzoyl, 4-dipropylaminomethylbenzoyl, 4-(morpholinomethyl)-benzoyl, 4-(4-methyl-1-piperazinylmethyl)-benzoyl, 3-dimethylamino-methylbenzoyl, 3 -diethylaminomethylbenzoyl, 3-dipropylaminomethylbenzoyl, 3-(morpholinomethyl)benzoyl, 3-(4-methyl-1-piperazinylmethyl)benzoyl, sulfo (in which case (I) designates a sulphate ester or a salt thereof) or phosphono (in which case (I) designates a phosphate ester or a salt thereof).

Preferred ethers of formula (I) are those wherein R¹⁰ is a methyl group, a methoxymethyl group, a benzyl group or a pivaloyloxymethyl group.

The naturally occurring compounds of the present invention can be obtained from natural sources by methods known per se. Alternatively, they may—like the structurally related synthetic sterols of the present invention—be obtained by synthesis by methods known per se.

The present invention is further illustrated by the following examples which, however, are not to be construed as limiting the scope of protection. The features disclosed in the foregoing description and in the following examples may, both separately and in any combination thereof, be material for realising the invention in diverse forms thereof.

### EXAMPLES

### Example 1

Isolation, Purification and Identification of a Meiosis Inducing Substance (MIS) from Bull Testes.

Testes from a six years old bull (Danish Landrace) were removed immediately after slaughter. The tunica albuginea was removed and the testicular tissue placed on dry ice and stored at −80° C. Frozen testicular tissue (92 g) was minced into pieces smaller than 1 mm³ and freeze dried in the dark until completely dry, approximately 90 h. The freeze dried tissue was extracted with 400 ml of n-heptane (LiChrosolv, Merck 4390, Germany) under nitrogen with stirring for 24 h at 20° C. The suspension was filtered and the solid material was extracted once more following the same procedure. The pooled organic phases were evaporated to dryness on a rotatory evaporator at room temperature yielding 981 mg of extracted material. This material was dissolved in n-heptane and portioned into 15 vials from which the n-heptane was evaporated. The vials were stored under nitrogen at 4° C. in the dark.

A three-step HPLC purification was employed for the extracts:

In the first step, the content of one vial was dissolved in 50 ml of 50% (v/v) tetrahydrofuran (THF) in water and applied to the reversed phase HPLC column (LiChroSpher 100 RP-8 endcapped 5 μm, 250×4 mmi.d., Merck). The elution was performed at 40° C. using a linear gradient of THF going from 50% to 100% in 15 min (flow: 1 ml/min). 18 fractions of 1 ml were collected and tested for MIS-activity.

In the second step, fractions from the first step which were found active in the oocyte-assay were dissolved in 50-100 ml of 70% THF and applied to a column similar to the one used in the first step. The elution was performed at 40° C. using a linear gradient of THF going from 60% to 78% in 16 min (flow: 1 ml/min). 8 fractions of 1 ml were collected and tested for MIS-activity.

In the third step, fractions from the second step which were found active in the oocyte-assay were dissolved in 100 ml of n-heptane:2-propanol (98:2) (v/v) and applied to a semipreparative HPLC column (ChromSpher Si 5 μm, 250×10 mm i.d., Chrompack). The elution was performed at room temperature using a mobile phase consisting of n-heptane:2-propanol, 98:2 (v/v) (flow: 5 ml/min). Five fractions of 2.5 ml were collected and tested for MIS-activity.

In all three steps, the elution was monitored by UV-detection at 220 nm

Material which had been through the three-step purification procedure described above was used to study the molecular structure of the active compound by nuclear magnetic resonance spectrometry (NMR) and by mass spectrometry.

For the NMR spectra, approximately 1 mg of purified material was dissolved in 0.6 ml of deuterochloroform. A ¹³C proton decoupled NMR spectrum, a ¹H NMR spectrum (with and without resolution enhancement) and a 2D TOCSY spectrum were recorded on a Bruker AMX2400 NMR spectrometer equipped with an inverse broad band 5 mm probe head with gradient coil. The ¹³C-NMR chemical shifts in ppm (d) for the isolated MIS are given in Table 1 for comparison with the corresponding data for zymosterol (Taylor, U. F. et al. J. Lipid Res. 22 (1981) 171) and lanosterol (Emmons, G. T. et al. Magn. Res. Chem. 27 (1989) 1012).

TABLE 1

| Carbon | Zymosterol | Lanosterol | MIS |
|---|---|---|---|
| 1 | 35.1 | 35.5 | 35.8 |
| 2 | 31.5 | 27.8 | 28.0 |
| 3 | 70.9 | 79.0 | 79.0 |
| 4 | 38.2 | 38.9 | 38.9 |
| 5 | 40.7 | 50.4 | 50.2 |
| 6 | 25.5 | 18.2 | 18.4 |
| 7 | 27.1 | 26.5 | 28.5 |
| 8 | 128.0 | 134.4 | 128.0 |
| 9 | 134.8 | 134.4 | 135.8 |
| 10 | 35.6 | 37.0 | 37.0 |
| 11 | 22.8 | 21.0 | 22.1 |
| 12 | 36.9 | 30.9 | 29.7 |
| 13 | 42.0 | 44.4 | 42.1 |
| 14 | 51.8 | 49.8 | 51.9 |
| 15 | 23.7 | 30.8 | 23.8 |
| 16 | 28.7 | 28.2 | 28.8 |
| 17 | 54.7 | 50.4 | 54.8 |
| 18 | 11.2 | 15.7 | 11.3 |
| 19 | 17.8 | 19.1 | 19.8 |
| 20 | 36.0 | 36.2 | 36.0 |
| 21 | 18.6 | 18.6 | 18.6 |
| 22 | 36.0 | 36.3 | 36.1 |
| 23 | 24.7 | 24.9 | 24.8 |
| 24 | 125.0 | 125.2 | 125.2 |
| 25 | 130.6 | 130.9 | 130.9 |
| 26 | 17.6 | 17.6 | 17.6 |
| 27 | 25.7 | 25.7 | 25.7 |
| 28 | | 15.4 | 15.4 |
| 29 | | 27.9 | 27.9 |
| 30 | | 24.2 | |

Mass spectrometry was performed using a VG Trio 1000 LC/MS instrument with LINC particle beam interphase and LAB-BASE 2.1 software (Fisons Instruments) with a HPLC system comprising a ChromSpher Si, 3 μm, 100×4.6 mm column (Chrompack). The HPLC was performed at room temperature and n-heptane:2-propanol, 98:2 (v/v) was used as mobile phase (flow: 0.6 ml/min). The sample of the MIS to be injected was dissolved in n-heptane. The mass spectrometer was operated in electron impact mode. Results are given in Table 2 in which the relative peak heights for the isolated product is compared to data for 4,4-dimethylzymosterol from Ref. 1. Under Ref. 2 a “+” designates that the corresponding peak was also reported in this study. A “−” under Ref. 1 or 2 designates that the corresponding peak was not reported in these studies.

TABLE 2

| m/z | Interpretation | MIS | Ref. 1 | Ref. 2 |
|---|---|---|---|---|
| 412 | [M]⁺ | 100 | 100 | + |
| 397 | [M—CH₃]⁺ | 60 | 42 | + |
| 379 | [M—CH₃—H₂O]⁺ | 24 | 17 | + |
| 301 | [M—SC]⁺ | 11 | − | + |
| 299 | [M—SC—2H]⁺ | 22 | 13 | − |
| 274 | [M—SC—C₂H₃]⁺ | 8 | 8 | − |
| 259 | [M—SC—C₃H_{6]}⁺ | 21 | 33 | + |
| 241 | [M—SC—C₃H₆—H₂O]⁺ | 44 | 33 | + |

SC=side chain, C₂H₃=position 16 and 17, C₃H₆=position 15, 16 and 17. Ref. 1: Baloch et al. Phytochemistry 23(1984)2219.Ref. 2: Morimoto et al. Liebigs Ann. Chem. 708(1967)230.

Based on the ¹³C-NMR spectrum and its molecular weight of 412 as determined by mass spectroscopy (MS), the structure of the MIS isolated from bull testes was proposed to be 4,4-dimethyl-^{5α}-cholesta-8,24-diene-^{3β}-ol, also designated 4,4-dimethylzymosterol (DMZ). The chemical shifts of the individual carbon atoms of the MIS-active material from the third HPLC purification step were compared with the chemical shifts of the structurally very closely related compounds lanosterol and zymosterol. The observed proton chemical shifts, coupling constants and TOCSY correlations fully support that the isolated compound is 4,4dimethylzymosterol.

### Example 2

Isolation, Purification and Identification of a Meiosis Inducing Substance (MIS) from Human Follicular Fluid.

Human follicular fluid (FF) was obtained from follicles aspirated for oocyte collection in the treatment of infertility by in vitro fertilization. The fluid was freeze dried and extracted with n-heptane and the extract was purified using the same procedures as described in Example 1. The compound of the active peak had a molecular ion of m/z=410 and the mass spectrum revealed that the chemical structure of the FF-MIS molecule is 4,4-dimethyl-^{5α}-cholesta-8,14,24-triene-^{3β}-ol.

Methods: Mass spectrometry was performed using a VG Trio 1000 LC/MS with LINC particle beam interphase and LAB-BASE 2.1 software (Fisons Instruments) connected to a straight phase HPLC system consisting of a ChromSpher Si, 3 μm, 100×4 mm i.d. column (Chrompack) and n-heptane:2-propanol:methanol:ammonia (68:30:2:0.2) as mobile phase (flow: 0.5 ml/min) at room temperature. The sample of the MIS to be injected was dissolved in n-heptane. The mass spectrometer was operated in electron impact mode. Results are shown in Table 3.

TABLE 3

| | |
|---|---|
| m/z | Interpretation |

| | | |
|---|---|---|
| 410 | = M | [M]⁺ (Mw for FF-MIS) |
| 395 | M-15 | [M-CH₃]⁺ |
| 392 | M-18 | [M-H₂O]⁺ |
| 377 | M-33 | [M-CH3-H₂O]⁺ |
| 349 | M-61 | [M-43-H₂O]⁺ |
| 381 | M-129 | [M-SC-H₂O]⁺ (SC = 111) |
| 279 | M-131 | [M-SC-2H-H₂O]⁺ |
| 257 | M-153 | [M-SC-42]⁺ |
| 255 | M-155 | [M-154-H]⁺ |
| 239 | M-171 | [M-SC-42-H₂O]⁺ |

SC = side chain

### Example 3

Preparation of 4^{β}-Methylzymosterol by Fermentation.

Step A: The yeast strain Kluyveromyces bulgaricus A3410 was inoculated on a YPG agarslant and grown for 3 days at 30° C. in a thermostated incubator. 5 ml of sterile YE medium was added to the slant and the yeast colonies were suspended in the liquid medium by shaking of the tube on a whirlimixer. The suspension of cells was then drawn up into a 5 ml sterile syringe and added to a 500 ml shakeflask with two baffle intrusions in the bottom. The flask contained 200 ml of ZYM medium. The flask was fixed on a rotating table and propagated for 24 hours at 250 rpm, 30° C. 0.4 ml of a sterile filtered amphotericin B solution was now added to the flask and the propagation was continued for further 25 hours. The yeast cells were harvested by centrifugation (Beckman model J6, 5° C., 10 min, 4000 rpm) and washed once in water. The cell slurry was isolated in a small plastic container and stored at −18° C. before the final extraction of the sterols.

The nutrient media and the amphotericin B solution mentioned above had the following composition:

| | |
|---|---|
| YPG agar | |
| Yeast extract, Difco | 4 g |
| KH₂PO₄ | 1 g |
| MgSO₄.7H₂O | 0.5 g |
| Glucose | 15 g |
| Agar | 20 g |
| Deionized water | 1000 ml |
| pH adjusted to 5.8 before autodavingat 121° C., 20 min. | |
| YE medium | |
| yeast extract, Difco | 10 g |
| Deionized water | 1000 ml |
| Autoclaving 121° C., 20 min. | |
| ZYM medium | |
| Yeast extract, Difco | 20 g |
| Peptone, Bacto | 10 g |
| Tap water | 1000 ml |
| pH adjusted to 6.5-6.6 before autoclaving 121° C., 20 min. | |
| Glucose (added separately | 60 g |
| after autoclaving) | |

Amphotericin B Solution

1 mg of Fungizone®(Lyophilized cake of 50 mg amphotericin B, 41 mg sodium deoxycholate and 20.2 mg sodium phosphate from Squibb) dissolved in 1 ml deionized water.

Step B: The cultured cells from step A were suspended in 10 ml of water and 10 ml of 40% KOH in methanol were added. The mixture was heated to reflux for 4 hours, left overnight at room temperature, and then extracted twice with 20 ml of n-heptane. The combined extracts were washed with 10% sodium chloride solution and then with water until neutral (five times) and dried. Evaporation of the solvent left 40 mg of crude sterols.

Step C: The crude sterols from step B were dissolved in 1 ml of n-heptane/2-propanol (98:2) and shaken on a vortex mixer, centrifuged at 5000 rpm for 10 min and then subjected to HPLC:

Column: LiChroSorb DIOL 10 μm, 250×4 mm i.d. (Merck)

Eluent: n-heptane/2-propanol (98:2)

Flow: 1.1 ml/min, 20° C.

Detection: UV at 220 nm

The peak eluting after 6.8 min was collected from several runs. The collected fractions were pooled and the solvent was evaporated to leave a residue which was submitted to mass spectrometry, and tested in the oocyte test.

The data of the mass spectrum which are reported in Table 4 are identical with those of 4^{β}-methylzymosterol as recorded in the National Bureau of Standards library.

TABLE 4

| | |
|---|---|
| m/z | Intepretation |

| | | |
|---|---|---|
| 398 | = M | M[M]⁺ (Mw of 4β-methy lzy mosterol) |
| 383 | M-15 | [M-CH₃]⁺ |
| 380 | M-18 | [M-H₂O]⁺ |
| 365 | M-33 | [M-CH₃-H₂O]⁺ |
| 269 | M-129 | [M-SC-H₂O]^{+ (SC = 111)} |
| 267 | M-131 | [M-SC-H₂O-2H]⁺ |
| 245 | M-153 | [M-SC-C₃H6]⁺ |
| 227 | M-171 | [M-SC-C₃H₆-H₂O]⁺ |
| 213 | M-185 | [M-SC-C₄H₈-H₂O]⁺ |

SC = Side Chain

### Example 4

Preparation of 4,4-Dimethylcholesta-8,14-Dien-^{3β}-ol.

This compound was prepared as described by Schroepfer et al.: Chemistry and Physics of Lipids 47(1988) 187, and showed physical constants as described in the literature.

### Example 5

Preparation of 4,4-Dimethylcholest-8-ene-^{3β}-ol.

Step A: 2.48 g of 4,4-dimethylcholesta-8,14-diene-3-ol (Example 4) was dissolved in 20 ml of pyridine at 0° C. 1.7 g of benzoyl chloride was added, and the mixture was stirred at ambient temperature overnight. After evaporation to dryness, 25 ml of toluene was added and after standard aqueous workup, evaporation and trituration with acetone, 2.3 g (74%) of crystal-line benzoate was obtained.

The ¹H-NMR spectrum (CDCl₃, δ) showed characteristic signals at: 8.1 (d,2H); 7.55 (t,1H); 7.4 (t,2H); 5.4 (s,broad,1H); 4.2 (dd,1H).

Step B: 2.04 g of 3-benzoyloxy-4,4-dimethylcholesta-8,14-diene (Step A) was dissolved in 50 ml of THF, and 360 ml of 1 M borane in THF was added dropwise at 0° C. The mixture was stirred at ambient temperature overnight, cooled to 0° C., and 140 ml of water was added dropwise, followed by 360 ml of 10% sodium hydroxide and 378 ml of 30% hydrogen peroxide. After stirring for 90 minutes, 100 ml of diethyl ether was added to the mixture and the aqueous phase extracted twice with diethyl ether. The combined organic phases were washed twice with sodium bisulphite solution and then with water. After evaporation, the product was purified by chromatography on SiO₂ (2% diethyl ether in toluene) to yield 0.62 g of 3-benzoyloxy-4,4-dimethylcholest-8-en-15-ol.

MS (molecular ion): 534.4.

The ¹H-NMR spectrum (CDCl₃, δ) showed characteristic signals at: 8.0 (d,2H); 7.5 (t,1H); 7.4(t,2H); 4.75 (m,1H); 4.1 (m,1H).

Step C: 0.54 g of 3-benzoyloxy-4,4-dimethylcholest-8-en-15-ol was dissolved in 2.7 ml of pyridine at 0° C. and 33 mg of dimethylaminopyridine and 287 mg of phenylchlorothioformate was added cautiously. The mixture was stirred for 20 hours at ambient temperature. After addition of25 ml of diethyl ether, the mixture was washed 6 times with a saturated solution of copper sulphate, water, twice with 10% sodium hydroxide, water and brine, and evaporated to yield 0.68 g of crude 3-benzoyl-4,4-dimethylcholest-8-ene-15-phenylthiocarbonate, which was further processed by dissolving in 20 ml of toluene and treated with 370 mg of tributyltin hydride and 20 mg of azo-isobutyronitril. The mixture was heated at 90° C. for 20 minutes, and the same treatment was repeated. After evaporation, the mixture was roughly purified by chromatography on SiO₂ (heptane/methylene chloride: 70/30) to yield 150 mg of crude 3-benzoyloxy-4,4-dimethylcholest-8-ene, contaminated with the corresponding 8,14diene (Step A).

Step D: 150 mg of the mixture prepared in Step C was dissolved in 2 ml of methylene chloride, cooled to 0° C. 0.7 ml of diisobutylaluminiumhydride was added dropwise and after 15 minutes, 0.15 ml of water was added cautiously. Then, 25 ml of diethyl ether was added, and the organic phase was washed twice with a saturated solution of potassium sodium tartrate, with water and with brine, and evaporated to yield 130 mg of a mixture that was chromatographed on AgNO₃/SiO₂ (prepared as described in: Chem. & Phys. of Lipids 63 (1992) 115) and eluted with toluene. Crystallisation from ether/methanol yielded 49 mg of the title compound.

Melting point: 154-155° C.

MS (molecular ion): 414.4.

The ¹³C-NMR spectrum (CDCl₃, 100.6 MHz) showed characteristic signals at 78.49 (C₃); 127.49 (C₈); 135.35 (C₉).

### Example 6

Preparation of 3-Acetoxy-4,4-Dimethylcholesta-8,14-Diene.

1.3 g of 4,4-dimethylcholesta-8,14-diene-3-ol (Schroepfer et al.: Chemistry and Physics of Lipids 47 (1988) 187) were dissolved in 7.5 ml of pyridine and 7.5 ml of acetic anhydride and stirred at 22° C. overnight. The mixture was evaporated in vacuo, stripped twice with toluene, and purified by flash chromatography on SiO₂ (toluene). The first 300 ml of eluate was evaporated, and the product crystallised from diethyl ether to yield 140 mg of 3-acetoxy-8,14-dimethylcholestadiene.

Melting point: 120-125° C. (with destruction).

MS (molecular ion): 454.4.

The ¹H-NMR spectrum (CDCl₃, δ) showed characteristic signals at: 5.4 (s,broad,1H); 4.5 (dd,1H); 2.0 (s,3H):

### Example 7

Preparation of Cholesta-8,14-diene-^{3β}-ol.

770 mg of dehydrocholesterol was dissolved in a mixture of 2.7 ml of benzene, 19 ml of ethanol and 2.7 ml of concentrated hydrochloric acid and heated at reflux temperature for 3 hours. The mixture was cooled in an ice bath whereby a first crop of 110 mg of crystals were obtained. Evaporation of the filtrate to dryness and crystallisation from ether/methanol gave a second crop of 220 mg of crystals, which was combined with the first crop and chromatographed on AgNO₃/SiO₂ (prepared as described in Example 5, step D) and eluted with 2.5% acetone in in toluene to yield 94 mg of pure cholesta-8,14-diene-^{3β}-ol.

Melting point: 113-114.5° C.

MS (molecular ion): 384.4.

The ¹H-NMR spectrum (CDCl₃, δ) of the product showed characteristic signals at: 5.35 (s,broad,1H); 3.6 (m,1H).

The ¹³C-NMR spectrum (CDCl₃, 50.3 MHz) showed characteristic signals at: 70.99(C₃); 117.42(C₁₅); 123.1(C₈); 140.8(C₉); 151.1(C₁₄).

### Example 8

Preparation of 4,4-Tetramethylenecholesta-8,14-Dien-3-ol.

Step A: 1.15 g of dehydrocholesterol was dissolved in 15 ml of 2-butanone, 0.34 g of aluminium isopropoxide was added, and the mixture was heated at reflux temperature for 75 minutes. After cooling on an ice bath, 15 ml of 2N hydrochloric acid was added, the phases were separated, and the organic phase was washed twice with 7.5 ml of 2N hydrochloric acid. The aqueous phase was extracted with toluene, and the combined organic phases were washed with water and brine, dried, and evaporated to yield 1.18 g of crude cholesta-5,7-diene-3-one as a viscous oil.

The ¹H-NMR spectrum showed characteristic signals at: 5.8(s,1H); 5.2(m,1H); 3.2(d,1H);2.7(dd,1H).

Step B: 0.67 g of potassium tert-butoxide was dissolved in 16 ml of tert-butanol at 45° C., 0.57 g of cholesta-5,7-diene-3-one was added, and the mixture was stirred for 10 minutes. 0.47 g of 1,4-diiodobutane was added, and the mixture was stirred for 30 minutes. The solvent was evaporated, the residue redissolved in toluene and water, and a little brine was added to induce separation of the phases. The organic phase was washed four times with water, and the combined aqueous phases were extracted once with toluene. The combined toluene extracts were dried and evaporated to yield 0.45 g of a foam which after crystallisation from diethyl ether/methanol yielded 0.35 g of crystalline 4,4-tetramethylenecholesta-5,7-diene-3-one.

MS (molecular ion): 436.4.

The ¹H-NMR spectrum (CDCl₃, δ) showed characteristic signals at 5.75 (d,1H); 5.5(m,1H).

Step C: 130 mg of LiAlH₄ was suspended in 6 ml of THF, and 1.97 g of 4,4-tetramethylenecholesta-5,7-diene-3-one dissolved in 40 ml of THF was added dropwise over 30 minutes. 15 minutes after the addition was completed there still remained some unreacted starting material (TLC), and an additional 65 mg of LiAlH₄ was added. After stirring for 30 minutes the reaction was complete, and 0.9 ml of water dissolved in 5 ml of THF was added dropwise. After 30 minutes stirring, excess of magnesium sulphate was added, and the mixture stirred for another 30 minutes, filtered and evaporated to dryness. The residue was dissolved in 25 ml of diethyl ether and 25 ml of methanol, and the ether was cautiously removed in vacuo. After stirring overnight, 1.75 g of crystalline 4,4-tetramethylene-cholesta-5,7-diene-3-ol was isolated by filtration.

MS (molecular ion): 438.4.

The ¹H-NMR spectrum showed characteristic signals at: 5.8(d,1H); 5.5(m,1H); 3.5(m,1H).

Step D: 770 mg of the compound prepared in step C was dissolved in a mixture of 2.38 ml of benzene, 17.5 ml of ethanol, and 2.38 ml of concentrated hydrochloric acid and heated at reflux for 16 hours, and evaporated in vacuo. The residue was redissolved in 5 ml of toluene, filtered, and chromatographed on a medium pressure column of AgNO₃/SiO₃ (heptane:toluene, 10:90) to yield 35 mg of 4,4-tetramethylene-cholesta-8,14-diene-3-ol.

MS (molecular ion): 438.4.

The ¹H-NMR spectrum (CDCl₃, δ) showed characteristic signals at 5.35 (s,broad,1H); 3.3(d,d,1H).

The ¹³C-NMR spectrum (CDCl₃, 100.6 MHz) showed characteristic signals at: 79.0(C₃); 117.4(C₁₅); 122.9(C₈); 141.3(C₉); 151.1(C₁₄).

### Example 9

Preparation of 4,4-Dimethylcholest-8(14)-ene-^{3β}-ol.

580 mg of 4,4-dimethylcholest-8-ene-^{3β}-ol was dissolved in 20 ml of diethylether and 20 ml of acetic acid. 60 mg of 10% Pd/C catalyst was added and the mixture was left with stirring overnight under hydrogen at 3.5 atm. The catalyst was removed, and the filtrate concentrated to 10 ml, whereby crystallisation started. 10 ml of methanol was added, and the crystals were collected after 16 hours. Recrystallisation from methanol yielded 230 mg of material, which was shown by ¹H- and ¹³C-NMR to be a mixture of the 8(9) and 8(14)-isomers.

The mixture was redissolved in 10 ml of diethylether and 10 ml of acetic acid. 75 mg of 5% Pt/C catalyst was added, and the mixture treated with hydrogen overnight at atmospheric pressure. The catalyst was removed, the solvent evaporated, and the crystalline residue triturated with 5 ml of methanol to yield 190 mg of pure 4,4-dimethylcholest-8(14)-ene-^{3β}-ol.

MS (molecular ion): 414.4

¹³C-NMR spectrum (CDCl₃, 100.6 MHz) shows characteristic signals at: 79.24(C₃); 126.11(C₈); 142.20(C₁₄)

### Example 10

Test of Meiosis Inducing Substances in the Oocyte Test.

Animals: Immature female mice (B6D2-F1, strain C57B1/2J) were kept under controlled lighting (14 hr light, 10 hr dark) and temperature, with food and water ad libitum. When the animals reached a weight of 13-16 grams (which correspond to the age of 20 to 22 days post partum) they were given a single injection (i.p.) of human menopausal gonadotropin (Humegon, Organon, The Netherlands) containing approximately 20 IU FSH and 20 IU LH (Ziebe, S. et al. Hum. Reprod. 8(1993) 385-88). 48 hours later the animals were killed by cervical dislocation.

Collection and culture of oocytes: The ovaries were removed, placed in HX-medium (se below) and freed of extraneous tissue. The collection- and culture medium consisted of Eagles minimum essential medium (Flow, USA), containing 4 mM hypoxanthine, 3 mg/ml of bovine serum albumin, 0.23 mM sodium pyruvate, 2 mM glutamine, 100 U/ml of penicillin, and 100 mg/ml of streptomycin (all Sigma, USA). This medium is termed HX-medium. The same medium but without HX was used as control medium.

The antral follicles of the ovaries were punctured under a dissecting microscope using a 27-gauge needle. Cumulus enclosed oocyte (CEO) of uniform size were selected and rinsed three times in fresh HX-medium.

Oocytes freed from cumulus cells, i.e. denuded oocytes, DO, were obtained by gentle flushing CEO through a fine-bore mouth-controlled pipet. CEO and DO were cultured in 4-well multidishes (Nunclon, Denmark) containing 0.5 ml of HX-medium except the controls which were cultured in control medium. Each well contained 35 to 50 oocytes. The test cultures were made with different concentrations of the compounds to be tested as indicated in Table 5.

The cultures were performed at 37° C. and 100% humidity with 5% CO₂ in air. The culture time was 24 hours.

Examination of oocytes: By the end of the culture period the number of oocytes with germinal vesicle (GV) or germinal vesicle breakdown (GVBD) and those with polar body (PB) was counted in an inverted microscope with differential interference contrast equipment. The percentage of oocytes with GVBD per total number of oocytes and the percentage of oocytes with PB per GVBD was calculated. The results for DO and CEO, calculated as units of MIS activity, are given in Table 5. One unit of MIS activity is defined as:

$$\frac{\% {GVBD}_{control} }{2}$$

and the number of MIS activity units is calculated as:

$$2 ( \frac{\% {GVBD}_{test} - \% {GVBD}_{control}}{\% {GVBD}_{control}} )$$

TABLE 5

| | | | Concentration, |
|---|---|---|---|
| Substance | DO | CEO | ^{μ}g/ml |
| 4,4-Dimethyl- | 2.7 | 2.9 | 1.2 |
| zy mosterol | 1.8 | 2.7 | 0.3 |
| | 0.6 | 1.3 | 0.2 |
| | 1.5 | 1.3 | 0.02 |
| | 0.9 | 1.0 | 0.002 |
| 4,4-Dimethyl-^{5α}-cholesta- | 0.6 | 2.3 | 0.3 |
| 8,14,24-triene-^{3β}-ol | 1.6 | 0.5 | 0.03 |
| Zymosterol | 1.2 | 1.1 | 0.1 |
| | 0.6 | 0.4 | 0.01 |
| | | 0.2 | 0.001 |
| ^{4β}-Methy lzy mo- | 6.2 | 3.5 | 3.9 |
| sterol | 0.8 | 2.4 | 0.13 |
| 4,4-Dimethylcholest- | 0.8 | 12.8 | 0.03 |
| 8-ene-^{3β}-ol | | | |
| 4,4-Dimethylcholesta- | 2.25 | 0 | 3.0 |
| 8,14-dien-^{3β}-ol | | | |

### Example 11

Test of Meiosis Inducing Substances in the Gonad Test.

The gonad test was performed essentially as described by Byskov, A. G. et al. Mol. Reprod. Dev. 34 (1993) 47-52. The results given in Table 6 were evaluated semiquantitatively as described by Westergaard, L. et al. Fertil. Steril. 41 (1984) 377-84.

TABLE 6

| | Concentration, | |
|---|---|---|
| Substance | ^{μ}g/ml | Result |
| 4,4-Dimethyl-zymosterol | 10 | ++ |
| 4,4-Dimethyl-^{5α}-cholesta-8,14,24-triene- | 30 | + |
| ^{3β}-ol | | |

### Example 12

Preparation of Mono (^{5α}-cholesta-8,14-dien)-^{3β}-Succinate.

0.50 g of ^{5α}-cholesta-8,14-dien-^{3β}-ol was dissolved in 10 ml of THF, followed by 0.39 g of succinic anhydride and 16 mg of 4-dimethylaminopyridine. The solution was heated at reflux overnight and then evaporated to dryness. The residue was suspended in 10 ml of water and the precipitate was filtered off and washed with water and dried to give 0.48 g of the title compound, which could be further purified by dissolving in a mixture of aqueous sodium hydrogen carbonate and ethanol, addition of hydrochloric acid to pH 2, followed by concentration of the solution to give precipitation.

Melting point: 128 -131° C.

MS (molecular ion): 484,4

The ¹H-NMR spectrum (CDCl₃, δ) of the product showed characteristic signals at: 5.36 (s,1H); 4.75 (m,1H); 2.67 (m,2H); 2.6 (m,2H).

The ^{13C}-NMR spectrum (CDCl₃, 100.6 MHz) showed characteristic signals at: 73.4; 117.1; 122.7; 140.0; 150.5; 171.2; 177.2.

### Example 13

Preparation of ^{3β}-Ethoxycarbonyloxy-^{5α}-cholesta-8,14-diene.

0.50 g of ^{5α}-cholesta-8,14-dien-^{3β}-ol was dissolved in a mixture of 5 ml of toluene and 5 ml of pyridine, while cooling in an ice bath. 2.3 ml. of ethylchloroformate dissolved in 5 ml of toluene was added over 5 min. After 30 min. the ice bath was removed and stirring was continued for 20 hours at room temperature and then 2 hours at 60° C. The reaction mixture was evaporated to dryness in vacuo and triturated with 10 ml of ethanol to give 0.505 g of the title compound, which could be further purified by recrystallisation from ethanol.

Melting point: 101-106° C.

MS (molecular ion): 456.3

The ¹H-NMR spectrum (CDCl₃, δ) of the product showed characteristic signals at: 5.30 (s,1H); 4.50 (m,1H); 4.12 (q,2H); 1.24 (t,3H).

The ¹³C-NMR spectrum (CDCl₃, 100.6 MHz) showed characteristic signals at: 62.6; 116.6; 122.2; 139.4; 150.0; 153.6.

### Example 14

Preparation of ^{3β}-Phosphonooxo-4,4-dimethyl-^{5α}-cholesta-8,14-diene.

2.00 g of 4,4-dimethyl-^{5α}-cholesta-8,14-dien-^{3β}-ol was dissolved in 10 ml of dry pyridine and added over 5 min. to a solution of 1.66 ml of phosphorus oxychloride in 10 ml of dry acetone while cooling in an ice bath. After stirring at room temperature for 30 min. the precipitate was filtered off and washed with dry acetone. The residue was suspended in 70 ml of water and heated at reflux for 1¼ hour. After cooling to room temperature, the precipitate was filtered off, washed with water and dried to give 0.93 g of crude product. 0.70 g of the crude product was dissolved in 75 ml 0.1 M aqueous potassium hydroxide and the solution was filtered through 10 g of Amberlite resin IR-120(H) and evaporated in vacuo to dryness. The residue was triturated with 10 ml of water and the precipitate was filtered off, washed with water and dried to give 0.48 g of the title compound.

Melting point: 183-185° C.

The ¹H-NMR spectrum (CDCl₃+2 drops of CD₃OD) of the product showed characteristic signals at: 5.36(s,1H); 3.89(m,1H).

The ¹³C-NMR spectrum (CDCl₃+2 drops of CD₃OD, 100.6 MHZ) of the product showed characteristic signals at: 85.1; 116.9; 1223; 140.9; 150.5.

### Example 15

Preparation of ^{3β}-Isonicotinoyl-^{5α}-Cholesta-8,14-diene.

0.50 g of ^{5α}-cholesta-8,14-dien-^{3β}-ol was dissolved in 5 ml of pyridine followed by 1.16 g of isonicotinoylchloride hydrochloride. The suspension was heated at reflux overnight, and then evaporated to dryness. The residue was suspended in 100 ml of water, while cooling in an ice bath. The precipitate was filtered off and washed with water and dried to give 0.97 g of the crude product, which was recrystallized from acetone/water to give 0.40 g of the title compound.

Melting point: 129-131° C.

The ¹H-NMR spectrum (CDCl₃, δ) of the product showed characteristic signals at: 8.77 (d,2H); 7.84 (d,2H); 5.39 (s, 1H); 4.49 (m,1H).

The ¹³C-NMR spectrum (CDCl₃, 50.3 MHz) showed characteristic signals at: 75.0; 117.7; 122.8; 123.3; 138.0; 140.3; 150.5; 150.9; 164.6.

### Example 16

Effect of LH, FF-MAS and Cholesterol on GVB in Isolated Perfused Ovaries from Immature Rats.

Animals and Experimental Procedure: Immature Sprague-Dawley rats (body weight: 50-55 g) were injected s.c. with 20 IU pregnant mare serum gonadotropin (PMSG). 48 hours later, the rats were anesthetized with a combination of Ketavet/Rompun (105 and 8.3 mg/kg BW, respectively). Heparin sulfate (300 IU) was injected into the tail vein.

Ovaries with connecting vasculature were surgically isolated and perfusions were performed in a recirculating system (Koos RD et al. (1984) Biol Reprod 30:1135-1141) containing approximately 35 ml of perfusion medium (Medium 199 with Earle's salts supplemented with 50 μg/ml gentamycin sulfate, 0.2 IU/ml insulin, and 4% BSA).

The ovaries were initially perfused for approximately 1 hour to allow metabolic stabilization of the tissue. Only the ovarian specimens which maintained a flow rate between 0.8 and 1.5 ml/min at pressure of 80 mmHg during the stabilization period were used in the experiments.

After 1 hour of preperfusion, the effect of 4,4-dimethyl-5^{α}-cholesta-8,14,24-triene-3^{β}-ol (FF-MAS), IBMX (iso-butyl-methyl-xanthine), LH (luteinizing hormone), and cholesterol were tested by dividing the ovarian specimens into control and experimental groups. Additions to the perfusion medium were made as follows:

TABLE 7

| | | | Cholesterol | |
|---|---|---|---|---|
| Group | FF-MAS μM | IBMX (mM) | (μM) | LH (^{μ}g/ml) |
| 1 | 0 | 0 | | |
| 2 | 10 | 0.2 | | |
| 3 | 30 | 0.2 | | |
| 4 | 60 | 0.2 | | |
| 5 | | 0.2 | 60 | |
| 6 | | 0.2 | | 0.1 |

Prior to addition to the perfusion medium, FF-MAS and cholesterol were diluted in ethanol, and LH and IBMX were diluted in perfusion medium. For the investigation of meiosis, perfusions were continued up to 12 h after addition of the compounds.

At termination of each perfusion ovaries were dissected free from adherent tissue and oocytes isolated under a stereo microscope by manual rupture of the follicles using a pair of 27 gauge needles. Oocytes per ovary were counted and examined using Nomarski differential interference contrast optics.

Oocytes with a germinal vesicle breakdown (GVB) were regarded as undergoing meiotic maturation (Tsafiri et al. (1980) J. Reprod. Fertil. 60:399-402). The % GVB, defined as percentage of oocytes undergoing GVB per total number of oocytes in each ovary was calculated. Each experimental group consisted of 7 perfused ovaries. Data are presented as mean±sd. Statistical differences were calculated by one-way ANOVA followed by Students's t-test. P-values<0.05 indicated a statistically significant higher % GVB in treated groups compared to the control.

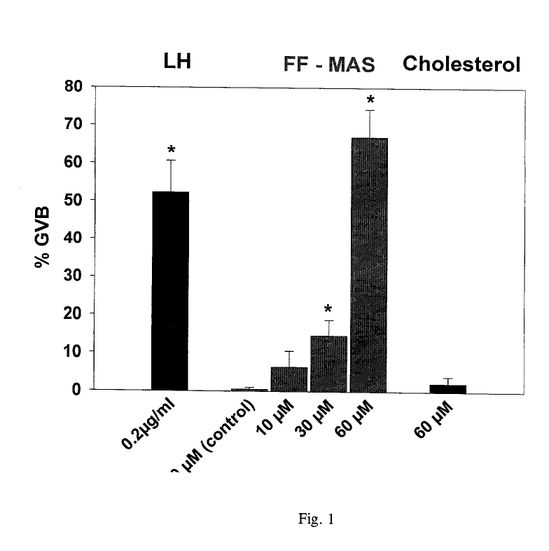

Results: The results show that FF-MAS was dose-dependently able to participate in the control of meiosis (FIG. 1). While perfused control ovaries did not show meiotic maturation (GVB: 0.5±0.48), 30 μM FF-MAS and 60 μM FF-MAS were able to significantly overcome the meiotic arrest resulting from treatment of the ovary with IBMX (control) alone. The results are statistically significant at the p<0.05 level. The effective dose of FF-MAS corresponds to 12-24 mg/liter bood volume. Based on the average blood volume content of a human subject of 5 liters ad an oral bioavailability of between 1-10%, an expected daily dosage can be calculated of approximately 1-10 g FF-MAS.

GVB for 30 μM FF-MAS was 14.6±4.2, and for 60 μM FF-MAS 66.9±7.4. 60 μM FF-MAS led to a comparable increase in GVB as was observed for LH (GVB: 52.4±8.2). In contrast, 60 μM cholesterol were not able to significantly induce meiotic maturation (GVB: 2.1±1.8).

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

Various references are cited herein, the disclosures of which are incorporated by reference in their entireties.

What is claimed is:

1. A method of regulating the meiosis in a mammalian germ cell in vivo, which method comprises administering to a mammal in need thereof a compound of the general formula (I) 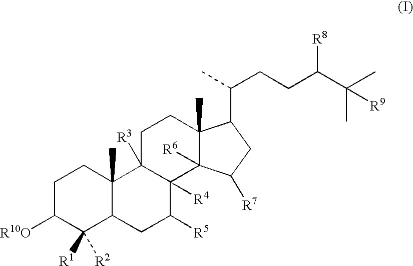
wherein
R¹ and R², independently, are selected from the group comprising hydrogen, unbranched or branched C₁-C₆ alkyl which may be substituted by halogen or hydroxy or wherein R¹ and R² together with the carbon atom to which they are bound form a cyclopentane ring or a cyclohexane ring;
R³ and R⁴ together designate an additional bond between the carbon atoms to which they are bound in which case R⁵ is hydrogen and R⁶ and R⁷ are either hydrogen or together they designate an additional bond between the carbon atoms to which they are bound; or
R⁵ and R⁴ together designate an additional bond between the carbon atoms to which they are bound in which case R³ is hydrogen and R⁶ and R⁷ are either hydrogen or together they designate an additional bond between the carbon atoms to which they are bound; or
R⁶ and R⁴ together designate an additional bond between the carbon atoms to which they are bound in which case R³, R⁵ and R⁷ are all hydrogen;
R⁸ and R⁹ are hydrogen or together they designate an additional bond between the carbon atoms to which they are bound; and
R¹⁰ is either hydrogen or an acyl group, a sulfo group or a phosphono group, or a group which together with the remaining part of the molecule forms an ether in an amount effective to regulate meiosis.

2. The method of claim 1 wherein R¹ is selected from the group consisting of hydrogen, methyl, ethyl, unbranched and branched C₃-C₆ alkyl, unbranched or a branched hydroxyalkyl group with up to six carbon atoms, unbranched or a branched α-hydroxyalkyl group with up to six carbon atoms, unbranched or a branched alkyl group substituted with halogen, and a trifluoromethyl.

3. The method of claim 1 wherein R² is selected from the group consisting of hydrogen, methyl, ethyl, unbranched and branched C₃-C₆ alkyl, unbranched or a branched hydroxyalkyl group with up to six carbon atoms, unbranched or a branched α-hydroxyalkyl group with up to six carbon atoms unbranched or a branched alklyl group substituted with halogen, and a trifluoromethyl.

4. The method of claim 1 wherein R¹ and R² together with the carbon atom to which they are bound form a cyclopentane ring or a cyclohexane ring.

5. The method of claim 1 wherein R³ and R⁴ together designate an additional bond between the carbon atoms to which they are bound and R⁵ is hydrogen.

6. The method of claim 1 wherein R⁵ and R⁴ together designate an additional bond between the carbon atoms to which they are bound and R³ is hydrogen.

7. The method of claim 1 wherein R⁶ and R⁴ together designate an additional bond between the carbon atoms to which they are bound and R³, R⁵ and R⁷ are hydrogen.

8. The method of claim 1 wherein R⁶ and R⁷ are hydrogen.

9. The method of claim 1 wherein R⁶ and R⁷ together designate an additional bond between the carbon atoms to which they are bound.

10. The method of claim 1 wherein R⁸ and R⁹ are hydrogen.

11. The method of claim 1 wherein R⁸ and R⁹ together designate an additional bond between the carbon atoms to which they are bound.

12. The method of claim 1 wherein R¹⁰ is hydrogen.

13. The method of claim 1 wherein R¹⁰ is an acyl group derived from an acid having from 1 to 20 carbon atoms.

14. The method of claim 1 wherein R¹⁰ is an acyl group selected from the group comprising acetyl, benzoyl, pivaloyl, butyryl, nicotinoyl, isonicotinoyl, hemi succinoyl, hemi glutaroyl, butylcarbamoyl, phenylcarbamoyl, butoxycarbonyl, tert-butoxycarbonyl and ethoxycarbonyl.

15. The method of claim 1 wherein R¹⁰ is an alkyl group, an aralkyl group, an alkyloxyakyl group or an alkanoyloxyalkyl group, each group comprising a total of up to 10 carbon atoms, preferably up to 8 carbon atoms, which together with the remaining part of the molecule forms an ether.

16. The method of claim 1 wherein R¹⁰ is a methoxymethyl group or a pivaloyloxymethyl group.

17. The method of claim 1 wherein R¹⁰ is sulfo.

18. The method of claim 1 wherein R¹⁰ is phosphono.

19. The method of claim 1 wherein the germ cell is an oocyte or a male germ cell.

20. The method according to claim 1, wherein the compound is 4,4-dimethylzymosterol.

21. The method according to claim 1, wherein the compound is 4,4-dimethyl-^{5α}-cholesta-8,14,24-triene-^{3β}-ol.

22. The method according to claim 1, wherein said mammal is administered between about 1 to about 10 g of said compound per day.
